(19) United States
(12) Patent Application Publication
(10) Pub. No.: US 2003/0056401 A1
(43) Pub. Date: Mar. 27, 2003

(54) Automatic inclination adjusting sole for golf shoes

(76) Inventor: Hae Boung Kwon, Ansan-shi (KR)

Correspondence Address: Welsh & Katz, Ltd., THOMAS R. VIGIL, 22nd Floor, 120 South Riverside Plaza, Chicago, IL, 60606, US

(21) Appl. No.: 10/274,507

(22) Filed: Oct. 17, 2002

Related U.S. Application Data
(63) Continuation of application No. PCT/KR01/00494, filed on Mar. 28, 2001.

(30) Foreign Application Priority Data
Apr. 17, 2000 (KR) 20 2000 0011180

(51) Int. Cl. A43B 5/00; A61F 5/14

(52) U.S. Cl. 36/127; 36/143

(57) ABSTRACT

A golf shoe is provided which facilitates a stable swing by causing a golfer to pull his knees inward securely. Because of the inwardly pulled knees, a gap is inevitably formed between the outer edge of fore shoe and ground surface. This gap usually causes unnecessary body movement during swing, and eventually leads to a loss of swing strength and accuracy (this is referred to as a sway). In order to prevent the sway by avoiding formation of the gap, a special golf shoe sole is invented. When a golfer is taking a stance with his knees pulled inward the inner part of the fore sole is lowered and the outer part of the fore sole is raised. Technical function of the sole is achieved by installing the inner (1) and outer (2) fluid chambers which are interconnected by the solution moving velocity control path (3). During swing, the slope at the fore part of the sole is adjusted optimally by itself by the pressure distribution of foot. During walking, the slope becomes flattened and usual comfortable walking is possible. With the present invention the swing stability is improved remarkably and at the same time a comfortable walking is possible. The invented sole can improve remarkably the swing performance when it is used in golf shoe.

### TECHNICAL FIELD

The present invention relates to a sole in golf shoe. In order to guarantee a stable swing, inclination (slope) on the sole is automatically adjusted by the pressure of golfer's foot. The slope is changed according to the golfer's stance and ground surface condition.

### BACKGROUND ART

Golfers are learning and practicing the basic posture in which an optimum distance between the feet is set and the knees are pulled inward 10 in order to achieve stable swing (see FIG. 1). Because the both knees are pulled inward, a gap is formed inevitably between the outer edge of golf shoe and ground surface as shown in FIG. 2, and the presence of this gap deteriorates swing stability. During swing, golfer's body moves uncontrollably first to one side and then to the other side, this in turn causes a loss of swing strength and accuracy (this is referred to as a sway in golf jargon). In the present invention, when a golfer is taking a stance for swing with his knees pulled inward, the slope of sole is adjusted to avoid the gap (see FIG. 3) by the foot pressure such that the inner part of the fore sole is lowered and the outer part is raised (see FIG. 4), and thus occurrence of the sway can be prevented. The present invention relates to a golf shoe which improves the swing performance, Purpose of the swing in golf is to fly the ball as far and accurately as possible, and the performance of swing has been improved significantly mainly by modifying the ball and clubs. However, an effort for improving the swing performance by modifying golf shoe has not been made. The present invention is made in an attempt to improve the swing stability by modifying the conventional golf shoe. Prior to explaining the golf boot invented in the present invention, features of swing made with conventional golf shoe are explained. Swing in golf comprises by a stance, address, back-swing, down-swing, impact, and finish. The stance means positioning the feet at proper distance from the ball, and the address indicates moving the club just behind the ball and taking a proper posture to hit the ball. Then, back-swing is followed, and it means lifting the club backward. The back-swung club is moved down to hit the ball, and this is called a down-swing. The down-moving club head bits the ball, and this is called an impact. The club head is moving upward after the impact to fully push the ball by taking an advantage of an inertia, and this is called a finish. In particular, in the back-swing step the club is moving backward and the weight center of golfer's body is likely to move right-hand side and the load by the body is usually placed on the right foot. If the body is moved outward due to the body weight, the strong down-swing and long range flying of ball can not be guaranteed. At the same time, the impact accuracy is also deteriorated. In order to prevent the outward movement of body during swing, the knees should be pulled inward as securely as possible when taking a stance and address (see FIG. 1). By pulling the knees inward the body is supported firmly by the right foot and can be kept at proper position without outward moving. It is noted that a gap is inevitably formed between the outer edge of fore boot and ground surface when the knees are pulled inward to take a good stance (see FIG. 2). During swing the center of body weight is moved on to the right foot, and the body is likely to move outward because of the gap. Therefore, a stable back-swing is hampered, and the impact accuracy is deteriorated inevitably. The gap also causes a deterioration in overall swing performance because after the impact the center of body weight is moved easily to the left foot and the eventually it is moved outward again due to the gap between the outer edge of the left boot and ground surface. In usual stance with a conventional golf shoe the feet are separated by proper distance and the knees are pulled inward securely, thus a gap between the outer edge of boot and ground surface is inevitable. The present invention is made in order to prevent the unstable swing due to the inevitable gap between the outer edge of shoe and ground surface usually caused by the conventional flat sole golf shoe.

### DISCLOSURE OF INVENTION

Technical Purpose This Patent is Aiming for

In the present invention, a smart device is installed in the fore part of sole of golf shoe, which automatically adjusts the slope of fore part of the sole by the pressure distribution of the golfer's foot. With this device, golfer can make an ideal back-swing, down-swing, impact, and finish by preventing the sway or by minimizing unnecessary body movement during swing. With the present invention the golfer can practice a long-range driving with enhanced accuracy. During golf swing, in particular, the back-swing, the center of golfer's body weight is placed on the right foot and then it moves to the left foot from the down-swing step. The ball is then impacted and finished properly to fly the ball far and accurately.

In all the basic steps of swing, the golfer's knees should be pulled inward (see FIG. 1) in order to prevent the sway in which the center of body weight moves uncontrollably. Because the both knees are pulled inward, a gap is formed inevitably between the outer edge of shoe and ground surface (see FIG. 2).

Because of this gap, the center of body weight is likely to move right- or left-side during the swing, and thus the sway is frequently caused and the hitting strength and accuracy are lost usually. This problem would be solved if a golfer wears shoe with particular sole, in which the height of inner part of sole is lower than the outer part. However, this sole can cause a discomfort during walking, which is usually as long as 7 km. In particular, the ground surface is not always flat and this irregular ground surface may cause a practical difficulty in taking stance and addressing with the shoe with the particular sole.

The present invention was made to prevent effectively the sway caused by the gap between the outer edge of shoe and ground surface (see FIG. 2).

The gap causes unnecessary movement of body during the swing. By removing the gap in the present invention, the movement of body is controlled properly and the stable swing can be achieved. In order to achieve a stable foot positioning when the both knees are pulled inward to take a stance and address for swing even on the uneven ground, the slope at the fore part of sole in shoe is adjusted automatically by changing the heights of inner and outer part of sole. When swinging with proper stance and address, the slope at fore part of sole in golf shoe is adjusted according to the shape of ground surface and address posture,

thus the gap between the outer edge of shoe and ground surface can be compensated (see FIG. 3). Thus, the unnecessary movement of body can be avoided, and the sway problem can be minimized. Optimum foot positioning depending on the ground surface condition can be achieved by the present invention.

### CONSTITUTION OF THE INVENTION

In order to achieve the technical goal, the slope at fore part of sole in shoe is adjusted automatically by changing the heights of outer and inner part of the fore sole. With the present invention the gap between the outer edge of shoe and ground surface is compensated (see FIG. 2).

The present invention consists of inner (1) and outer (2) fluid chambers and fluid moving path(s) connecting the two fluid chambers (see FIG. 4). The inner (1) and outer (2) fluid chambers are located at inner and outer part of the fore sole, respectively. When a pressure is applied to the chambers by foot, the fluid can move from the inner chamber (1) to the outer chamber (2) through the fluid moving path (3), and vice versa, according to the pressing situation. Thus, the height of the chambers is changed according to the foot pressure situation and the slope at the fore part of sole can be adjusted to an optimum condition for ideal swing. The fluid moving path controls the moving velocity of fluid. When a golfer takes a stance and pulls his both knees inward, the pressure of inner part of fore foot is applied on the inner fluid chamber (1). Then, the fluid moves from the inner chamber (1) to the outer chamber (2), and its velocity is so fast that optimum slope for ideal stance on the sole is achieved instantly. At this state, the inner fluid chamber (1) is under squeezed condition and the outer fluid chamber (2) is under expanded condition. The quick slope formation is made possible by opening the check valve installed at the fluid moving path. During the swing, displacement of the center of body weight will exert a pressure on the outer fluid chamber (2) through the foot. Under this situation, however, the fluid in the outer fluid chamber (2) will not move to the inner fluid chamber (1) by closing slowly the check valve. Therefore, the optimum slope achieved on taking stance will be maintained all the way to the completion of the swing, and ideal swing can be performed. For controlling the velocity of fluid movement through the path, ball-type or plate-type check valve (4) is installed at the path.

When a golfer wearing the shoe invented in present invention takes a stance on flat ground with his knees pulled inward as shown in FIG. 1, the inner part of fore foot will exert a pressure on the inner part of the fore part of sole. Thus the inner fluid chamber (1) will be squeezed and the fluid will be moved to the outer fluid chamber (2). What is important at stage is that the fluid in the inner chamber should move quickly to the outer chamber, so that the inner part of fore sole will be lowered and the outer part will be raised quickly. Thanks to this configuration, an optimum slope at the fore part of sole will be formed as soon as the golfer takes a stance (see FIG. 3). During swing, a change in the distribution of pressure under the foot will, of course, occur in such a way that the center of body weight is moved to the outer part of fore foot and this exerts a pressure on the outer fluid chamber (2). Fluid in the outer chamber (2) is, then, willing to move to the inner chamber (1). However, the fluid movement from the outer chamber to inner chamber is limited. The fluid movement from the outer chamber to the inner chamber is controlled to take place very slowly by the fluid moving velocity control path (3). Thus the optimum slope on the sole can be maintained until whole swing is completed. Meanwhile, during walking after finishing the swing, the fluid in the both chambers move mutually to make a balance and the slope achieved at the beginning of swing will be disappeared and a comfortable flat sole will be restored for comfortable walking.

Golf shoe designed in the present invention can improve the swing performance even on the ground with slope where the position of left foot is lower respect to the right foot. Under this circumstance, the gap between the outer edge of left shoe and ground surface is enlarged When a golfer wearing the invented shoe is taking a stance with his knees pulled inward, the pressure from the left foot will be exerted mainly on the inner fluid chamber (1) in the left shoe, and the fluid will move from the inner chamber (1) to the outer chamber (2) in the sole. Thus, height of the inner part of fore foot will be lowered and the outer part will be raised, so that the unfavourable slope at fore foot will be improved and an even and comfortable foot position will be obtained. For the right foot, the inner chamber will be raised and the outer chamber is lowered when taking a stance and address on the ground with the slope. Thus, a comfortable foot positioning can be obtained for stable swing.

When a golfer takes a stance on the ground with slope where the right foot is placed at the position lower than the left foot (see FIG. 6), the gap between the outer edge of right boot and ground surface will be enlarged. When a golfer wearing the invented boot is taking a stance with his knees pulled inward, pressure of the right foot will be exerted on the inner fluid chamber (1) in the right shoe, and the fluid will move from the inner chamber (1) to the outer chamber (2) in the sole. Thus, the height of inner part of the fore foot will be lowered and the outer part will be raised, so that the unfavourable ground slope will be compensated and an even and comfortable foot position will be obtained.

### EFFECT OF THE INVENTION

Golf swing is performed through a series of actions of back-swing, down-swing, impact, and finish, and it is very important that the movement of body during the swing should be sustained. In order to sustain the body movement and thus to achieve powerful and accurate swing, the golfer pulls his knees inward on stance. Because of the knees pulled inward, a gap between the outer edge of shoe and ground surface is formed inevitably (see FIG. 2). This gap causes a movement of the body weight center to the right foot during back-swing, and then the center of body weight is moved form the right foot to the left foot during the following stages from down-swing to finish. This movement of the body weight center usually causes an unnecessary body movement during swing. This unnecessary body movement is the cause of sway, which leads to a loss of strength and accuracy of swing.

With the present invention, the slope at fore part of sole in golf shoe is adjusted automatically and optimally for ideal swing. The gap caused by pulling the knees inward can be compensated by the slope formed at the fore part of sole. Therefore, the slope of sole in shoe is adjusted optimally according to the ground condition, and each steps in swing can be accomplished in optimum condition. Meanwhile, during walking the slope of sole is adjusted such that a usual comfortable walking is possible by changing the slope by itself. With the present invention a golfer can practice a perfect golf swing by the self-adjustable sole slope. The optimally adjusted slope can guarantee a stabilized swing by preventing the sway.

### BRIEF DESCRIPTION OF THE DRAWINGS

FIG. 1 shows the basic swing posture of a golfer wearing conventional golf shoe with his knees pulled inward.

FIG. 2 shows the gap between the outer edge of shoe and ground surface caused by pulling the knees inward for a conventional golf shoe.

FIG. 3 shows disappearance of the gap for the golf shoe invented in the present invention. The gap between the outer edge of shoe and even ground surface is compensated by the slope change in sole.

FIG. 4 shows the fluid chambers installed in the sole of right foot. Cross-section of the fluid chambers is also included.

FIG. 5 shows the adjusted slope in the invented shoe on the ground with slope where the left foot is located at lower position than the right foot.

FIG. 6 shows the adjusted slope in the invented shoe on the ground with slope where the right foot is located at lower position than the left foot.

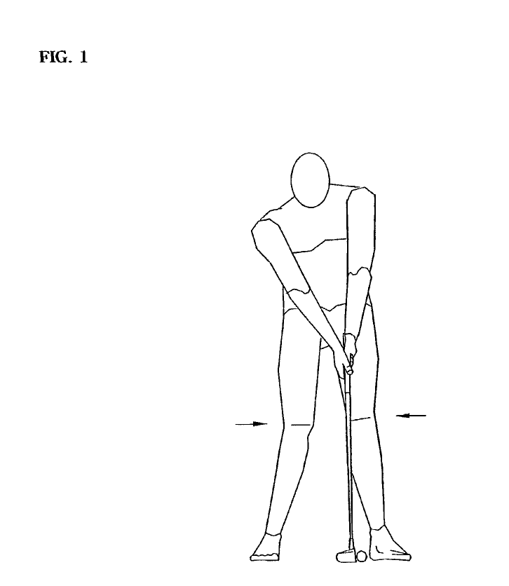

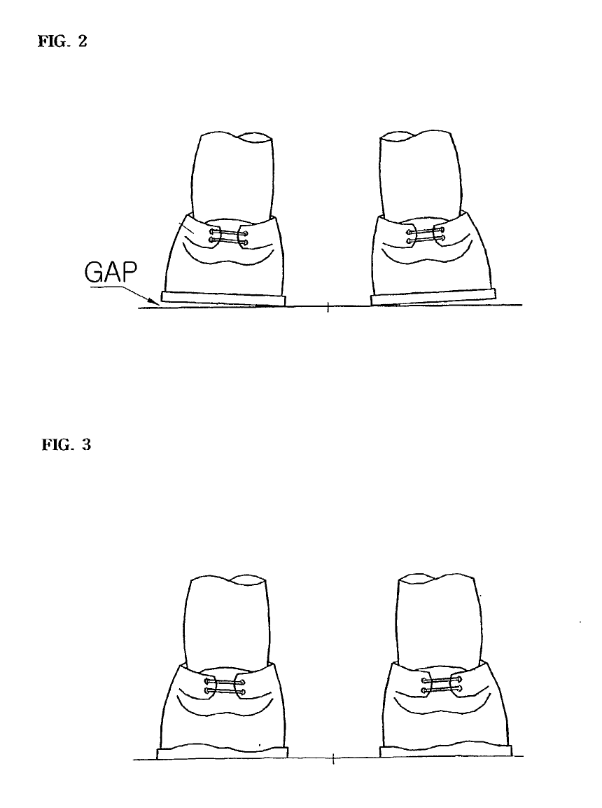

### EXPLANATION OF THE NUMBER DENOTING VARIOUS PARTS IN THE DRAWING

1: inner fluid chamber

2: outer fluid chamber

3: fluid moving velocity controlling path

4: check valve

### BEST MODE FOR CARRYING OUT THE INVENTION

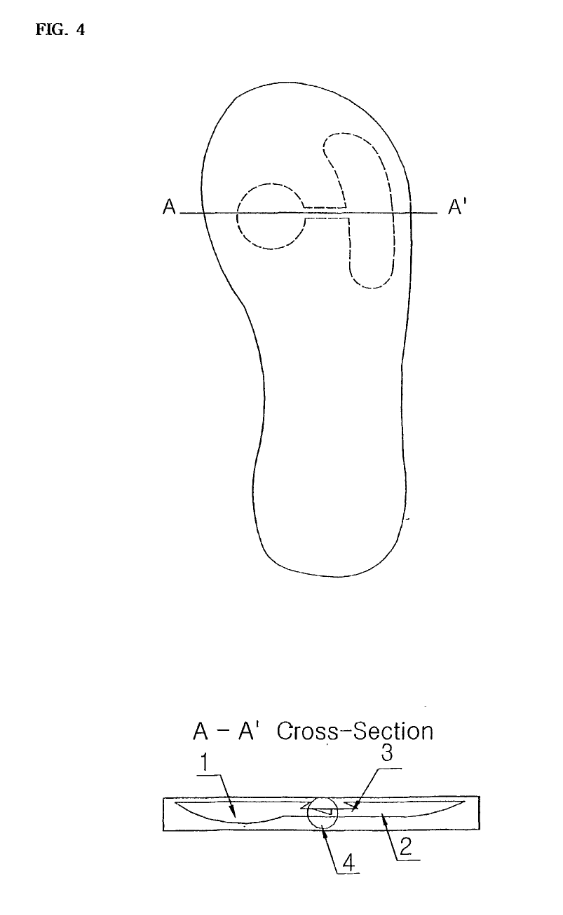

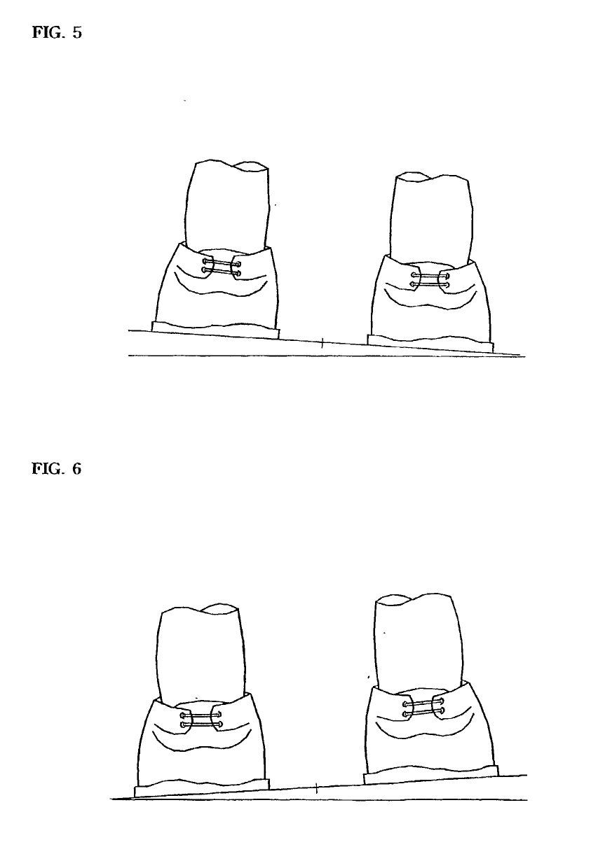

In the present invention, as shown in FIG. 4, the inner (1) and outer (2) fluid chambers are installed in the fore part of the sole in golf shoe, and the two chambers are interconnected by the fluid moving velocity controlling path (3). When a golfer is taking a stance, the body weight exerts a pressure on the inner fluid chamber, thus the height of inner chamber (1) is lowered (inner chamber is squeezed).

At the same time, the outer chamber (2) is raised (outer chamber is expanded). Thus, the slope at fore part of sole is adjusted by itself and a stabilized swing is available. The present invention is featured with the check valve (4) installed at the fluid moving velocity control path (3), which controls the velocity of fluid movement between the two chambers. With the check valve the movement of fluid from the inner chamber to the outer chamber is taking place very rapidly. On the contrary, the fluid movement from the outer chamber to the inner chamber is taking place very slowly. Therefore, the optimum slope beneath the fore part of foot achieved at the early stage of swing can be maintained until swing is fully completed. The slope of fore part of foot is adjusted easily by the pressure from foot during swing. During walking the slope at the fore part of sole is flattened by itself and a comfortable walking is possible. With the present invention, therefore, the swing stability is improved remarkably and at the same time the usual comfortable walking is also achieved.

I claim:

1. Sole of golf shoes comprising:
an inner fluid chamber installed at inner part of the sole;
an outer fluid chamber installed at outer part of the sole; and
a fluid moving velocity control path disposed between the inner fluid chamber and the outer fluid chamber, wherein cross sectional area of the fluid moving velocity control path is controlled by a check valve, so that fluid moves quickly from the inner chamber to the outer chamber and slowly from the outer chamber to the inner chamber.

2. Sole of golf shoes comprising:
an inner fluid chamber installed at inner part of the sole;
an outer fluid chamber installed at outer part of the sole; and
a fluid moving velocity control path disposed between the inner fluid chamber and the outer fluid chamber, wherein cross sectional area of the fluid moving velocity control path is determined for optimum slope on the sole.
